(19) United States
(12) Patent Application Publication
(10) Pub. No.: US 2025/0188134 A1
(43) Pub. Date: Jun. 12, 2025

(54) POLYPEPTIDE, FUSION-TYPE MULTIMERIC PROTEIN AND USES THEREOF

(71) Applicant: SUNRESIN NEW MATERIALS CO. LTD., Xi'an, Shaanxi (CN)

(72) Inventors: Yanjun LI, Xi'an (CN); Gang LI, Xi'an (CN); Tongnian GU, Xi'an (CN); Jiantao ZHANG, Xi'an (CN); Long LIU, Xi'an (CN); Xiaoju LEI, Xi'an (CN); Qiong LIU, Xi'an (CN)

(21) Appl. No.: 18/557,208

(22) PCT Filed: Dec. 21, 2022
(86) PCT No.: PCT/CN2022/140536

§ 371 Date: Oct. 25, 2023

(30) Foreign Application Priority Data
Sep. 1, 2022 (CN) CN202211064221.6

(51) Int. Cl. C07K 14/31; B01D 15/38; B01J 20/24; C07K 1/22

(52) U.S. Cl. CPC C07K 14/31; B01D 15/3809; B01J 20/24; C07K 1/22; C07K 2319/705

(57) ABSTRACT

The present invention relates to the technical field of protein biological functions, in particular to a polypeptide, a fusion type multimeric protein and use thereof, the polypeptide is selected from: (1), a polypeptide having a substitution mutation in at least one position selected from the group consisting of positions 16, 25, 29, 49 and 58, as compared with a natural C structural domain of a protein A as shown in SEQ ID NO.1; wherein, the position 16 is subjected to a substitution mutation into leucine or valine; the position 25 is subjected to a substitution mutation into lysine, arginine, histidine or tryptophan; the position 29 is subjected to a substitution mutation into alanine, leucine or threonine; the position 49 is subjected to a substitution mutation into arginine or histidine; and the position 58 is subjected to a substitution mutation into glycine, isoleucine or alanine; or (2) a polypeptide having at least 80% of sequence identity to the polypeptide in (1) and retaining substitution mutation in at least one position selected from the group consisting of positions 16, 25, 29, 49 and 58; the polypeptide has high alkali tolerance and high loading capacity.

### CROSS REFERENCE TO RELATED APPLICATIONS

The present application claims priority to Chinese Patent Application No. 202211064221.6, entitled “POLYPEPTIDE, FUSION-TYPE MULTIMERIC PROTEIN AND USES THEREOF”, and filed to the China National Intellectual Property Administration on Sep. 1, 2022, the entire contents of which are incorporated herein by reference.

### TECHNICAL FIELD

The present invention relates to the technical field of protein biological functional technology, in particular to a polypeptide, a fusion-type multimeric protein and uses thereof.

### BACKGROUND

As of February 2022, the FDA has approved a total of 109 antibody drugs, and the global antibody drug market has maintained a growth rate of over 10% for 8 consecutive years. In 2021, it exceeded $200 billion for the first time, with an increase of 16.5% compared to 2020. The separation of antibodies is crucial in the production of antibody drugs, and protein A affinity purification is a key step in antibody separation and purification. Currently, protein A fillers are sensitive to alkaline conditions, and the elution conditions are too strict. With the continuous expansion of antibody purification scale in the market, the requirements for their loading capacity and alkaline resistance performance are becoming higher and higher.

Staphylococcal Protein A (SPA), also known as Protein A, is a cell wall protein of Staphylococcus aureus. In 1983, the SPA gene was cloned and expressed in Escherichia coli by Duggleby et al. (Duggleby, C. J and Jones, SA: cloning and expression of the Staphylococcus aureus protein A gene in Escherichia coli. Nucl Acids Res. 11(1983) 3065-3076; Lofdahl, S., Guss, B., et al; Gene for Staphylococcal protein A. Proc. Natl. Acid. Sci. USA 80(1983) 697-701). SPA protein molecule is composed of five highly homologous structural domains E, D, A, B and C, and X structural domain, with a total of six structural domains. Because the five highly homologous structural domains E, D, A, B and C of SPA have the ability to bind to IgG and can bind to the Fc and Fab regions of most mammalian IgG (Jansson B, Uhlén M, Nygren PA. All individual domains of staphylococcal protein A show Fab binding. FEMS Immunol Med Microbiol. 1998 January; 20(1):69-78.). Therefore, SPA, as an affinity ligand for antibody purification coupled to solid-phase carrier, is widely used in the separation and purification of antibodies.

Due to the presence of impurities such as lipids, nucleic acids, proteins, polysaccharides, cell fragments, or contamination by viruses and bacteria in the sample during the antibody production process, these impurities may be non-specific adsorbed by protein A affinity fillers during the purification of antibodies, thereby contaminating the fillers. In addition, the protein A chromatography column also requires Clean In Place (CIP). In order to improve the service life of protein A filler and ensure column efficiency during its use, it is necessary to clean and regenerate the filler. The most widely used solution currently is to clean the chromatographic column in place with 0.1-1 M NaOH. However, natural protein A is intolerant to NaOH at concentrations higher than 0.1 M (Garshasb, Rigi, Samira, et al. A comprehensive review on staphylococcal protein A (SpA): Its production and applications. [J]. Biotechnology & Applied Biochemistry, 2019.), the development of genetic engineering has gradually improved the alkali resistance of recombinant protein A, but the high consumption and limited service life in industrial production of antibody drugs have led to increased production costs. Therefore, protein A fillers with higher loading capacity and higher alkali resistance are an inevitable trend pursued by antibody manufacturers and the development direction of filler production enterprises.

Antibodies are generally composed of Fab and Fc structural domains. The interaction between the Fab region of the antibody and SPA can seriously affect the elution pH value, leading to the need for lower pH values to achieve IgG dissociation and elution. The B structural domain mutant obtained by replacing glycine at position 29 of the B structural domain of the SPA molecule with alanine has almost no interaction with the Fab region. When asparagine at position 23 of the B structural domain is replaced by threonine, the alkali resistance of B structural domain mutant was significantly improved (Amritkar V, Adat S, Tejwani V, et al. Engineering Staphylococcal Protein A for high-throughput affinity purification of monoclonal antibodies [J]. Biotechnology advances, 2020:107632.). However, the modified SPA molecules are still unable to meet the current demand for higher loading capacity and higher alkali resistance of SPA molecules.

### SUMMARY OF THE INVENTION

Therefore, the technical problem to be solved in the present application is to provide a polypeptide, a fusion-type multimeric protein and uses thereof. The obtained fusion-type multimeric protein has higher loading capacity and higher alkali resistance.

Therefore, the present application provides the following technical solution:

According to one aspect of the present application, the present application provides a polypeptide, wherein the polypeptide is selected from:
(1) a polypeptide having a substitution mutation in at least one position selected from the group consisting of positions 16, 25, 29, 49 and 58, as compared with a natural C structural domain of a protein A as shown in SEQ ID NO.1; wherein,
the position 16 is subjected to a substitution mutation into leucine or valine;
the position 25 is subjected to a substitution mutation into lysine, arginine, histidine or tryptophan;
the position 29 is subjected to a substitution mutation into alanine, leucine or threonine;
the position 49 is subjected to a substitution mutation into arginine or histidine; and
the position 58 is subjected to a substitution mutation into glycine, isoleucine or alanine; or
(2) a polypeptide having at least 80% of sequence identity to the polypeptide in (1) and retaining substitution mutation in at least one position selected from the group consisting of positions 16, 25, 29, 49 and 58.

Optionally, the position 16 is subjected to a substitution mutation into leucine; and/or
the position 25 is subjected to a substitution mutation into lysine; and/or
the position 29 is subjected to a substitution mutation into alanine; and/or
the position 49 is subjected to a substitution mutation into arginine; and/or
the position 58 is subjected to a substitution mutation into glycine.

Optionally, the polypeptide has the substitution mutation in position 58, and has the substitution mutation in at least one position selected from the group consisting of positions 16, 25, 29 and 49; or
the polypeptide has the substitution mutation in position 49, and has the substitution mutation in at least one position selected from the group consisting of positions 16, 25, 29 and 58; or
the polypeptide has the substitution mutation in position 29, and has the substitution mutation in at least one position selected from the group consisting of positions 16, 25, 49 and 58; or
the polypeptide has the substitution mutation in position 25, and has the substitution mutation in at least one position selected from the group consisting of positions 16, 29, 49 and 58; or
the polypeptide has the substitution mutation in position 16, and has the substitution mutation in at least one position selected from the group consisting of positions 25, 29, 49 and 58; or
optionally, the polypeptide has substitution mutations at positions 16, 25, 29, 49 and 58; or
optionally, the polypeptide has substitution mutations at positions 16, 25, 49 and 58; or
optionally, the polypeptide has substitution mutations at positions 49 and 58; or
optionally, the polypeptide has substitution mutations at positions 29, 49 and 58; or
optionally, the polypeptide has substitution mutations at positions 16, 25 and 29; or
optionally, the polypeptide has substitution mutations at positions 16 and 25; or
optionally, the polypeptide has a substitution mutation at position 49; or
optionally, the polypeptide has a substitution mutation at position 25.

According to another aspect of the present application, the present application provides a fusion-type multimeric protein, wherein the fusion-type multimeric protein comprises the fusion-type multimeric protein formed by fusion of the polypeptide.

The fusion-type multimeric protein further comprises polypeptide B and/or polypeptide Zmb; wherein
the polypeptide B satisfies any of the following conditions:
a. having an amino acid sequence as shown in SEQ ID NO.2; and
b. having an amino acid sequence of at least 80% sequence identity to the amino acid sequence as shown in SEQ ID NO.2 in a; the polypeptide Zmb satisfies any of the following conditions:
c. inserting an amino acid sequence ML in at least one position of the amino acid sequence shown in SEQ ID NO.2 in a, wherein the amino acid sequence of the amino acid sequence ML is shown in SEQ ID NO.3; and
d. inserting an amino acid sequence ML in at least one position of amino acid sequence described in b;
optionally, the amino acid sequence of the polypeptide Zmb is shown in SEQ ID NO.4.

The fusion-type multimeric protein, wherein the polypeptide is designated polypeptide Cm; the fusion-type multimeric protein comprises at least one polypeptide Cm, and/or at least one polypeptide Zmb;
optionally, the fusion-type multimeric protein comprises 1 to 6 polypeptides Cm, and/or 1 to 4 polypeptides Zmb;
optionally, the fusion-type multimeric protein comprises 4 polypeptides Cm and/or 2 polypeptides Zmb.

The fusion-type multimeric protein, further comprises at least one functional polypeptide FLD, wherein the at least one functional polypeptide FLD satisfies any of the following conditions:
A. having an amino acid sequence shown in SEQ ID NO.5; and
B. having an amino acid sequence of at least 80% sequence identity to the amino acid sequence as shown in SEQ ID NO.5 in A;
optionally, the functional polypeptide FLD is located between the polypeptide Cm and the polypeptide Zmb;
optionally, the fusion-type multimeric protein comprises 4 polypeptides Cm, 1 functional polypeptide FLD and 2 polypeptides Zmb sequentially from N-terminal to C-terminal.

According to another aspect of the present application, the present application provides a biomaterial, wherein the biomaterial comprises any of the following:
1) a nucleic acid molecule encoding the polypeptide or the fusion-type multimeric protein; optionally, the nucleic acid molecule is DNA or RNA;
2) an expression cassette, a recombinant vector, a recombinant microorganism or a transgenic cell line expressing the polypeptide or the fusion-type multimeric protein;
3) an expression cassette, a recombinant vector, a recombinant microorganism or a transgenic cell line containing the nucleic acid molecule described in 1);
4) a recombinant vector, recombinant microorganism or transgenic cell line containing the expression cassette described in 2) or 3); and
5) a host cell containing the recombinant vector described in 2) or 3) or 4);
optionally, the recombinant vector is constructed from the Escherichia coli expression vector pET-30a(+);
optionally, the host cell is Escherichia coli, and optionally, the Escherichia coli is Escherichia coli BL21(DE3).

Use of the polypeptide or the fusion-type multimeric protein in antibody detection, separation or purification.

According to another aspect of the present application, the present application provides an affinity chromatography medium, wherein the affinity chromatography medium comprises the fusion-type multimeric protein and an affinity chromatography medium vector;
optionally, the affinity chromatography medium vector includes agarose gel, dextran, cellulose, high molecular polymer with hydroxyl groups or silica gel.

The technical solution of the present application has the following advantages:

1. The present application provides a polypeptide, wherein the polypeptide is selected from: (1), a polypeptide having a substitution mutation in at least one position selected from the group consisting of positions 16, 25, 29, 49 and 58, as compared with a natural C structural domain of a protein A as shown in SEQ ID NO.1; wherein, the position 16 is subjected to a substitution mutation into leucine or valine; the position 25 is subjected to a substitution mutation into lysine, arginine, histidine or tryptophan; the position 29 is subjected to a substitution mutation into alanine, leucine or threonine; the position 49 is subjected to a substitution mutation into arginine or histidine; and the position 58 is subjected to a substitution mutation into glycine, isoleucine or alanine; or (2) a polypeptide having at least 80% of sequence identity to the polypeptide in (1) and retaining substitution mutation in at least one position selected from the group consisting of positions 16, 25, 29, 49 and 58. The polypeptide used has high alkali resistance and high loading capacity, resulting in higher loading capacity and alkali resistance of the fusion-type multimeric protein prepared using this polypeptide.

2. The fusion-type multimeric protein provided in the present application comprises the fusion-type multimeric protein formed by fusion of the polypeptide, and has higher loading capacity and higher alkali resistance.

3. The fusion-type multimeric protein provided in the present application also comprises polypeptide Zmb, and the amino acid sequence of the polypeptide Zmb is shown in SEQ ID NO.4. The experimental results show that when the polypeptide Zmb structural domain is used to prepare filler for purifying antibodies, the pH value of the elution buffer is increased from 3.3 to 4.3.

4. The fusion-type multimeric protein provided in the present application also comprises at least one functional polypeptide FLD. The insertion of the FLD reduces the steric hindrance effect of the three-dimensional structure of the fusion type polymer protein, enabling it to recombine to IgG, thereby improving the overall loading capacity of the fusion-type multimeric protein.

5. The affinity chromatography medium provided in the present application comprises the fusion-type multimeric protein and an affinity chromatography medium vector. The fusion-type multimeric protein can be coupled with a chromatography medium vector to form an affinity chromatography medium for antibody detection, separation, or purification.

### BRIEF DESCRIPTION OF THE DRAWINGS

To describe the technical solutions in specific implementations of the present invention or the prior art more clearly, the following briefly introduces the accompanying drawings required for describing the specific implementations or the prior art. Apparently, the accompanying drawings in the following description show some implementations of the present invention, and a person of ordinary skill in the art may still derive other drawings from these accompanying drawings without creative efforts.

FIG. 1 is the SDS-PAGE gel electrophoresis result of BL21-pET30a-CmZmb bacterial fluid in Example 5 of the present application; in the figure, lane 1 is before lactose induction; lane 2 is lactose induced; lane 3 is the lysate supernatant 1; lane 4 is the lysate supernatant 2; lane 5 is the lysate supernatant 3; lane 6 is eluent 1; and lane 7 is eluent 2; lane 8 is a protein marker (provided by Shaanxi Proanti Biotechnology Development Co., Ltd., Article No.: 10225-1);

FIG. 2 is the results of purified protein of BL21-pET30a-CmZmb bacterial fluid in Example 5 of the present application; in the figure, lane 1 is the lysate supernatant 2; lane 2 is a flowing liquid; lane 3 is eluent; lane 4 is 4% B solution for eluting impurities; lane 5 is 8% B solution for eluting impurities; lane 6 is 40% B solution for eluting target protein; and lane 7 is a 100% B solution for eluting target protein.

FIG. 3 shows the dynamic loading capacity test results of the fusion-type multimeric protein in Experiment Example 1 of the present application;

FIG. 4 shows the dynamic loading capacity test results of the alkali resistance of the fusion-type multimeric protein in Experiment Example 2 of the present application.

### DETAILED DESCRIPTION

The following examples are provided in order to further understand the present invention better, are not limited to the best implementation mode, and do not limit the content and protection scope of the present invention, anyone under the inspiration of the present invention or use the present invention Any product identical or similar to the present invention obtained by combining features of other prior art falls within the protection scope of the present invention.

If no specific experimental steps or conditions are indicated in the examples, it can be carried out according to the operation or conditions of the conventional experimental steps described in the literature in this field. The reagents or instruments used, whose manufacturers are not indicated, are all commercially available conventional reagent products.

The coding gene of polypeptides and fusion-type multimeric protein in the following examples were synthesized by Nanjing GenScript Biotechnology Co., Ltd.

In the present invention, amino acid residues can be the following abbreviations: lysine (K), glycine (G), alanine (A), arginine (R), glutamic acid (E), isoleucine (I), leucine (L).

### Example 1: Polypeptide Cm

1. In order to explore the alkali resistance solution of the polypeptide Cm, a series of polypeptide Cm are designed in this example, and the details of the polypeptide are as follows:

Polypeptide C (natural): the amino acid sequence is shown in SEQ ID NO.1.

Polypeptide Cm (K58G): compared with the natural C structural domain of protein A shown in SEQ ID NO.1, the polypeptide only has a substitution mutation of lysine (K) at position 58 to glycine (G).

Polypeptide Cm (K49R): compared with the natural C structural domain of protein A shown in SEQ ID NO.1, the polypeptide only has a substitution mutation of lysine (K) at position 49 to arginine (R).

Polypeptide Cm (G29A): compared with the natural C structural domain of protein A shown in SEQ ID NO.1, the polypeptide only has a substitution mutation of glycine (G) at position 29 to alanine (A).

Polypeptide Cm (E25K): compared with the natural C structural domain of protein A shown in SEQ ID NO.1, the polypeptide only has a substitution mutation of glutamic acid (E) at position 25 to lysine (K).

Polypeptide Cm (116L): compared with the natural C structural domain of protein A shown in SEQ ID NO.1, the polypeptide only has a substitution mutation of isoleucine (I) at position 16 to leucine (L).

Polypeptide Cm (K58G, K49R, G29A, E25K, I16L): compared with the natural C structural domain of protein A shown in SEQ ID NO.1, the polypeptide has:
a substitution mutation of lysine (K) at position 58 to glycine (G);
a substitution mutation of lysine (K) at position 49 to arginine (R);
a substitution mutation of glycine (G) at position 29 to alanine (A);
a substitution mutation of glutamic acid (E) at position 25 to lysine (K); and
a substitution mutation of isoleucine (I) at position 16 to leucine (L).

Polypeptide Cm (K58G, K49R, E25K, I16L): compared with the natural C structural domain of protein A shown in SEQ ID NO.1, the polypeptide has:
a substitution mutation of lysine (K) at position 58 to glycine (G);
a substitution mutation of lysine (K) at position 49 to arginine (R);
a substitution mutation of glutamic acid (E) at position 25 to lysine (K); and
a substitution mutation of isoleucine (I) at position 16 to leucine (L).

Polypeptide Cm (K58G, K49R): compared with the natural C structural domain of protein A shown in SEQ ID NO.1, the polypeptide has:
a substitution mutation of lysine (K) at position 58 to glycine (G); and
a substitution mutation of lysine (K) at position 49 to arginine (R).

Polypeptide Cm (K58G, K49R, G29A): compared with the natural C structural domain of protein A shown in SEQ ID NO.1, the polypeptide has:
a substitution mutation of lysine (K) at position 58 to glycine (G);
a substitution mutation of lysine (K) at position 49 to arginine (R); and
a substitution mutation of glycine (G) at position 29 to alanine (A).

Polypeptide Cm (G29A, E25K, 116L): compared with the natural C structural domain of protein A shown in SEQ ID NO.1, the polypeptide has:
a substitution mutation of glycine (G) at position 29 to alanine (A);
a substitution mutation of glutamic acid (E) at position 25 to lysine (K); and
a substitution mutation of isoleucine (I) at position 16 to leucine (L).

Polypeptide Cm (E25K, 116L): compared with the natural C structural domain of protein A shown in SEQ ID NO.1, the polypeptide has:
a substitution mutation of glutamic acid (E) at position 25 to lysine (K); and
a substitution mutation of isoleucine (I) at position 16 to leucine (L).

Further, the substitution mutation at position 16 involved in the above polypeptide Cm can also be a substitution mutation into valine;
the substitution mutation at position 25 can also be a substitution mutation into arginine or histidine or tryptophan;
the substitution mutation at position 29 is a substitution mutation into leucine or threonine;
the substitution mutation at position 49 is a substitution mutation into histidine; and
the substitution mutation at position 58 is a substitution mutation into isoleucine or alanine.

### Example 2

In this example, the polypeptide Zmb is designed, and the polypeptide Zmb is as follows:

Polypeptide B: having an amino acid sequence as shown in SEQ ID NO.2;

Polypeptide Zmb: inserting an amino acid sequence ML between position 20 and position of the amino acid sequence shown in SEQ ID NO.2, wherein the amino acid sequence of the amino acid sequence ML is shown in SEQ ID NO.3; that is the amino acid sequence of the polypeptide Zmb is shown in SEQ ID NO.4.

### Example 3

In this example, a functional polypeptide FLD is designed, and the functional polypeptide FLD has an amino acid sequence as shown in SEQ ID NO.5.

### Example 4: Fusion-Type Multimeric Protein

In this example, a series of fusion-type multimeric proteins were designed using the polypeptides of Examples 1-3, as follows:
CC: an amino acid sequence fused with six polypeptide C (natural) sequentially from N-terminal to C-terminal, that is, the arrangement is: C-C-C-C-C-C.
CmCm (K58G): an amino acid sequence fused with six polypeptides Cm (K58G) sequentially from the N-terminal to the C-terminal.
CmCm (K49R): an amino acid sequence fused with six polypeptides Cm (K49R) sequentially from the N-terminal to the C-terminal.
CmCm (G29A): an amino acid sequence fused with six polypeptide Cm (G29A) sequentially from the N-terminal to the C-terminal.
CmCm (E25K): an amino acid sequence fused with six polypeptides Cm (E25K) sequentially from the N-terminal to the C-terminal.
CmCm (116L): an amino acid sequence fused with six polypeptides Cm (116L) sequentially from the N-terminal to the C-terminal.
CmCm (K58G, K49R, G29A, E25K, 116L): an amino acid sequence fused with six polypeptide Cm (K58G, K49R, G29A, E25K, 116L) sequentially from the N-terminal to the C-terminal.
CmCm (K58G, K49R, E25K, 116L): an amino acid sequence fused with six polypeptide Cm (K58G, K49R, E25K, 116L) sequentially from N-terminal to C-terminal.
CmCm (K58G, K49R): an amino acid sequence fused with six polypeptides Cm (K58G, K49R) sequentially from the N-terminal to the C-terminal.
CmCm (K58G, K49R, G29A): an amino acid sequence fused with six polypeptide Cm (K58G, K49R, G29A) sequentially from the N-terminal to the C-terminal.
CmCm (G29A, E25K, I16L): an amino acid sequence fused with six polypeptide Cm (G29A, E25K, 116L) sequentially from the N-terminal to the C-terminal.
CmCm (E25K, I16L): an amino acid sequence fused with six polypeptides Cm (E25K, 116L) sequentially from the N-terminal to the C-terminal.
CDB: an amino acid sequence fused from N-terminal to C-terminal by 4 polypeptides C (natural), 1 functional polypeptide FLD, and 2 polypeptides B. The arrangement is: C-C-C-C-FLD-B-B.
CB: from the N-terminal to the C-terminal, the amino acid sequence is fused with 4 polypeptides C (natural) and 2 polypeptides B, and the arrangement is: C-C-C-C-B-B.
CmZmb: from the N-terminal to the C-terminal, the amino acid sequence is fused with 4 polypeptides Cm, 1 functional polypeptide FLD, and 2 polypeptides Zmb. The arrangement is: Cm-Cm-Cm-Cm-FLD-Zmb-Zmb. Its amino acid sequence is shown in SEQ ID NO.6, and its gene sequence is shown in SEQ ID NO.7.

Further, the substitution mutation at position 16 involved in the above polypeptide in the above-mentioned fusion-type multimeric protein can also be a substitution mutation into valine;
the substitution mutation at position 25 can also be a substitution mutation into arginine or histidine or tryptophan;
the substitution mutation at position 29 is a substitution mutation into leucine or threonine;
the substitution mutation at position 49 is a substitution mutation into histidine; and
the substitution mutation at position 58 is a substitution mutation into isoleucine or alanine.

### Example 5: Preparation of Fusion-Type Multimeric Protein

In this example, the fusion-type multimeric protein designed in Example 4 is prepared, and the preparation process is as follows:

### (1) Construction of Recombinant Vector

The coding gene the fusion-type multimeric protein and the expression vector pET-30a(+) were double enzyme digested by NdeI (purchased from Shenggong Biotechnology (Shanghai) Co., Ltd., B600120) and HindIII (purchased from Shenggong Biotechnology (Shanghai) Co., Ltd., B600184), and the corresponding fragments were recovered and connected (DNA gel recovery kit, purchased from Shanghai Biyuntian Biotechnology Co., Ltd., D0056). The connected product was mixed with the top 10 receptive cells of Escherichia coli, and was placed on ice for 30 min, heat-shocked in the water bath at 42° C. for 90 s, placed on ice for transformation 3 min, 100 μl room temperature LB liquid medium was added, and cultured in a shaker at 37° C. at 220 rpm for 60 min, the bacterial fluid was mixed well and applied to kanamycin (purchased From Shengong Bioengineering (Shanghai) Co., Ltd., A506636) resistant plate, the plate was inverted, cultured overnight at 37° C., and the transformant with kanamycin resistance was screened, thereby screening to obtain a recombinant vector expressing the fusion-type multimeric protein. For example, the recombinant vector expressing the fusion-type multimeric protein CmZmb obtained by screening is named pET-30a-CmZmb. Similarly, the recombinant vectors for other fusion-type multimeric proteins were named.

### (2) Construction of Recombinant Bacteria

Fresh BL21 (DE3) bacterial fluid was ice-bathed for 30 min, 4° C., centrifuged at 1000 g for 10 min, and the precipitate was resuspended with 0.1 mol/L MgCl₂—CaCl₂) (80 mmol MgCl₂ and 80 mmol CaCl₂, in which MgCl₂ and CaCl₂ were purchased from Sinopharm Chemical Reagent Co., Ltd., analytical pure, and the rest chemical reagents have the same purity), 4° C., centrifuged at 1000 g for 10 min, then resuspended in 0.1 mol/L CaCl₂ solution to prepare BL21 (DE3) competent cells, the recombinant vector (such as pET-30a-CmZmb) screened in step (1) into competent cells of Escherichia coli BL21 (DE3), the transformant was screened on the LB plate containing kanamycin, and it was verified to be a recombinant transformant by sequencing verify, named BL21-pET30a-CmZmb. Similarly, the recombinant transformants for other fusion-type multimeric proteins were named.

### (3) Fermentation of Recombinant Bacteria

The recombinant transformants (such as BL21-pET30a-CmZmb) with correct sequencing were inoculated in LB medium with an inoculum volume of 0.5% (volume percentage), cultured overnight at 37° C. until the OD₆₀₀ reached 0.8, and lactose with a final concentration of 10 g/L was added for inducing, bacteria was collected by centrifugation after 3-5 hours;

10 μl of 5× loading buffer (provided by Shaanxi Proanti Biotechnology Development Co., Ltd., Article No.: 10137-1) was added to 40 μl of bacterial fluid (pre lactose induced bacterial fluid and post lactose induced bacterial fluid, respectively) and boiled for 5 min, sample loading 20 μL to carry out SDS-PAGE gel electrophoresis, and the results were shown in FIG. 1. It can be seen that a new protein band appears at the position of 44 kD in the lactose-induced BL21-pET30a-CmZmb bacterial liquid (see FIG. 1 lane 2), the BL21-pET30a-CmZmb bacterial fluid without lactose induction does not appear this band or this band is lighter in color (see FIG. 1 lane 1). This demonstrated that the fusion-type multimeric protein CmZmb was induced to express in BL21-pET30a-CmZmb, similarly, other fusion-type multimeric protein in Example 4 were also successfully induced to express.

### (4) Purification

Analytical pure sodium chloride was added to a solution containing 20 mM PB (prepared using 5.8018 g/L of disodium hydrogen phosphate dodecahydrate aqueous solution and 0.5928 g/L of disodium hydrogen phosphate dihydrate aqueous solution, with standard buffer preparation method) to a concentration of 11.688 g/L. The pH was adjusted to 7.4 using an acid-base aqueous solution to obtain buffer solution A. The bacterial liquid collected in the above step (3) was suspended with the buffer A liquid according to the weight ratio of 1:10, the lysis time was 20 min, 30 min, 40 min respectively, ultrasonically crushed, and the supernatant was collected after centrifugation at room temperature to obtain the supernatant of the lysate supernatants 1, 2 and 3. Simultaneously, the lysate supernatant 2 was subjected to nickel column affinity purification (according to the following lysate supernatant 2 (lysis time 30 min) to carry out the nickel column affinity purification operation until elution with 40% B solution, and the eluate was collected and divided into two parts, i.e. eluent 1 and eluent 2). SDS-PAGE electrophoresis detection shows that the results are shown in lanes 3-7 in FIG. 1 (the samples using three lysis times show no difference in results, see lanes 3-5 in FIG. 1, and the lysate supernatant 1 in lane 3 corresponds to a lysis time of 20 min, the lysate supernatant 2 in lane 4 corresponds to a lysis time of 30 min, and the lysate supernatant 3 in lane 5 corresponds to a lysis time of 40 min. The results of eluent 1 and eluent 2 show no significant difference, see lanes 6-7 in FIG. 1), most of the fusion-type multimeric protein was expressed in the periplasm of E. coli, and the expression amount in the precipitate was less.

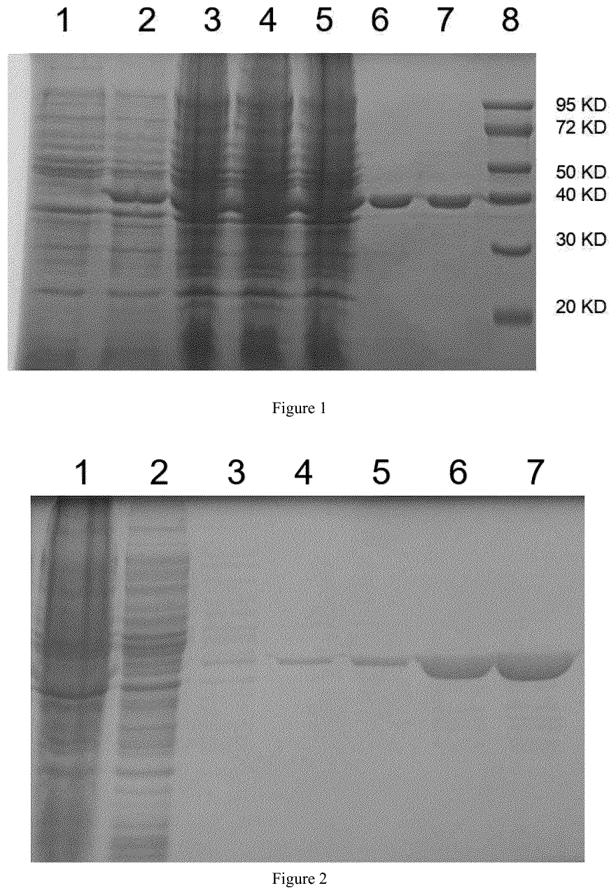

The lysate supernatant 2 (lysis time 30 min) was subjected to nickel column affinity purification, the specific purification steps: the flow rate of the purifier (AKTA, Purifier) was adjusted to 3 ml/min, the column was equilibrated with buffer A to make A280 nm (detection wavelength), A231 nm (detection wavelength) and A215 nm (detection wavelength) baselines are leveled by about 10 CV, and the baseline was flat; the sample was loaded through the A pump (lysate supernatant 60-120 ml); the chromatographic column was washed with buffer A and flattened to the baseline by about 10 CV, the impurities was washed with 8% (percentage by volume) of B solution (B solution is buffer A with a final concentration of 17.02 g/L imidazole, which was purchased from Sinopod Chemical Reagents Co., LTD.), and flatten to the baseline about 10 CV; 40% (volume percentage) B solution was used for eluting the target protein, and the baseline was about 10 CV; B solution (100% (volume percentage)) was used for eluting about 3 CV, until the baseline was flat (the absorption of protein at A280 nm was very small, the absorption of protein at A215 nm was high, but imidazole has a great influence on A215 nm, when collecting protein, it is necessary to repeatedly confirm the collection range, when the concentration was low, and the collection should start at the peak of A215 nm or A231 nm when the concentration is low and when the concentration is high, it can be collected according to the peak of A280 nm); the chromatographic column and the chromatographic system were washed with buffer A until the baseline flat is about 10 CV. The purified recombinant proteins with characteristic peaks, such as the fusion-type multimeric protein CmZmb with a purity of more than 80% (the results are shown in the swimming lane in FIG. 2) were collected, and similarly, the purity of other fusion-type multimeric proteins can also reach more than 80%.

### Example 6: Preparation of Affinity Chromatography Medium

This example provides the preparation of affinity chromatography medium, as follows:

The fusion-type multimeric protein obtained in Example 5 was used to prepare affinity chromatography media respectively, and the specific steps were as follows: 2-30 mL (15 ml was selected in this example) cyanogen bromide was added to 100 g of Sepharose 4FF (SUNRESIN NEW MATERIALS CO. LTD.), reacted at −10° C. to 10° C. (0° C. was selected in this example) for 15 to 30 min (22 min was selected in this example), 2 to 50 mL (25 ml was selected in this example) triethylamine was added, and reacted at −10° C. to 10° C. (0° C. was selected in this example) for 15 to 60 min (35 min was selected in this example), washed with acetone; 0.2 to 2 g (1 g was selected in this example) fusion-type multimeric protein was dissolved with pure water, was added to above-mentioned agarose gel 4FF, pH was adjusted to 7 to 11 (pH=8 was selected in this example), reacted at 0-20° C. (10° C. was selected in this example) overnight, 0.1-10% (volume percentage, 5% in this example) ethanolamine solution of pH 7 to 11 was added, reacted overnight at 0° C. to 40° C. (20° C. was selected in this example) to obtain an affinity chromatography medium. The prepared affinity chromatography medium is stored in 20% (volume percent) ethanol solution.

### Experimental Example 1: Dynamic Capacity Test of Fusion-Type Multimeric Protein Filler

The ÄKTA pure chromatography system programming was used to carry out the experiment, the chromatography column medium was the affinity chromatography medium prepared in Example 6, 1.1 ml affinity chromatography medium was mixed well with 20% (volume percentage) 2 ml of ethanol aqueous solution, constant flow pump (purchased from Shanghai Qingpu Huxi Instrument Factory) was used to loaded the chromatography column medium into the chromatographic column at a flow rate of 8 rpm/min, the detection wavelength was set to 280 nm, the pre-column pressure was 0.3 MPa, the chromatographic column was connected to the system, and the flow rate was set to 1.0 ml/min, rinse 10 CV with mobile phase B1, equilibrate 15 CV with mobile phase A1; load sample (2 mg/ml IgG, purchased from Beijing Suo Laibao Technology Co., Ltd., SP031) flow rate 0.2 ml/min, load sample to the absorb value at 280 nm is 90 mAu; constant elution mobile phase was set to 100% (volume percentage) mobile phase B1, flow rate was set to 1.0 ml/min, eluted to baseline flat, the eluent was collected; Finally, 10 ml was regenerated using mobile phase A1 at a flow rate of 1.0 ml/min. The absorption value is dynamically detected and monitored from the start to the end of the program
Mobile phase A1: 0.15M NaCl containing 20 mm PB, pH 7.4;
Mobile phase B1: 0.1M citric acid-sodium citrate buffer, pH3.3;
Dynamic load=(V_{A}×C₀)/V_{C}(mg/ml gel)
C₀ is the concentration of the target protein in the sample; V_{A} is the total volume of the sample loaded when the concentration of the target sample in the breakthrough curve reaches 5% CO; V_{C} is the total column bed volume (see Liu Ying, Zeng Jiancheng, Yu Miao, et al. A method for determining the dynamic loading capacity of an affinity chromatography filler: CN 114280208 A [P].)

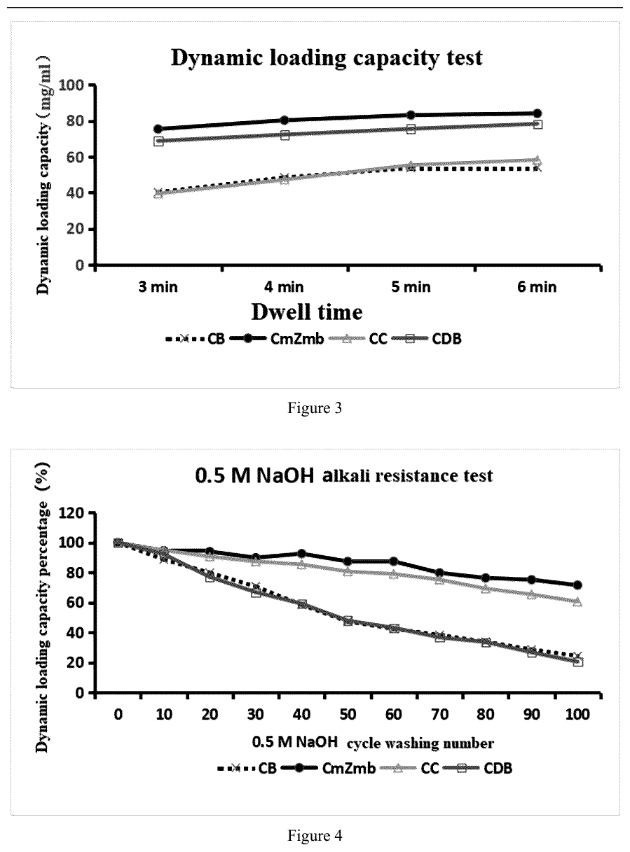

The dynamic loading capacity of fillers prepared with CmZmb, CC, CDB, and CB (peptide C in CC, CDB, and CB are all polypeptide C (natural)) was tested using the above method. The results are shown in FIG. 3, and the dynamic loading capacity of fillers prepared with CmZmb is higher than that of fillers prepared with CC, CDB, and CB.

Implement according to the above-mentioned method, when elution buffer pH4.3 (pH of mobile phase B1 is 4.3), the elution efficiency of CmZmb, CC, CDB and CB were respectively 86.6%, 56.8%, 87.9% and 62.3%. The affinity chromatography medium prepared by CmZmb has a relatively higher efficiency in eluting the target protein with a pH 4.3 buffer compared to other affinity chromatography media.

### Experimental Example 2: Alkali Resistance Test of Fusion-Type Multimeric Protein Filler

The ÄKTA pure chromatography system programming was used to carry out the experiment, the chromatography column medium was the affinity chromatography medium prepared in Example 6, 1.1 ml affinity chromatography medium was mixed well with 20% (volume percentage) 2 ml of ethanol aqueous solution, constant flow pump (purchased from Shanghai Qingpu Huxi Instrument Factory) was used to loaded the chromatography column medium into the chromatographic column at a flow rate of 8 rpm/min, the detection wavelength was set to 280 nm, the pre-column pressure was 0.3 MPa, the chromatographic column was connected to the system, and buffer A2 was used to equilibrate at a flow rate of 0.5 mL/min for 10 min, wash off the ethanol in the column, wash the column with 0.5M NaOH at a flow rate of 0.2 mL/min for 15 min, then wash the column with buffer A2 at a flow rate of 0.5 mL/min for 10 min, the above step was repeated 100 cycles, and the dynamic load was determined after every 10 cycles (see Experimental Example 1 for the calculation method of the dynamic loading capacity). Buffer A2: 0.15M NaCl containing 20 mM PB, pH7.4.

The alkali resistance test results of the fusion-type multimeric protein prepared only from polypeptide C (native) or polypeptide Cm were shown in the table below, partly shown in FIG. 4.

TABLE 1
Alkali resistance test results of fillers prepared
from fusion-type multimeric protein

| | | Dynamic | Dynamic |
|---|---|---|---|
| Fillers | Initial | loading | loading |
| prepared from | dynamic | capacity | capacity |
| fusion-type | loading | after 100 | percentage |
| multimeric | capacity | cycles | after |
| protein | (mg/ml) | (mg/ml) | 100 cycles (%) |
| CC | 58.26 | 35.27 | 60.54 |
| CmCm (K58G) | 41.29 | 24.18 | 58.56 |
| CmCm (K49R) | 57.69 | 36.91 | 63.98 |
| CmCm (G29A) | 45.69 | 29.99 | 65.64 |
| CmCm (E25K) | 42.98 | 27.07 | 62.98 |
| CmCm (I16L) | 48.47 | 28.58 | 58.96 |
| CmCm (K58G, K49R, | 56.02 | 41.31 | 73.74 |
| G29A, E25K, I16L) | | | |
| CmCm (K58G, K49R, | 55.68 | 37.13 | 66.69 |
| E25K, I16L) | | | |
| CmCm (K58G, K49R) | 46.78 | 29.16 | 62.34 |
| CmCm (K58G, K49R, | 55.94 | 38.18 | 68.25 |
| G29A) | | | |
| CmCm (G29A, E25K, | 49.68 | 33.18 | 66.78 |
| I16L) | | | |
| CmCm (E25K, I16L) | 48.56 | 29.73 | 61.23 |

2. The alkali resistance test results of the fillers prepared by CmZmb, CC, CDB and CB were shown in FIG. 4. It can be seen from the figure that the percentage of dynamic loading in the alkali resistance test of the fillers prepared by CmZmb is higher than that of CC, CDB and CB.

Obviously, the foregoing embodiments are merely examples for clear description, rather than a limitation to implementations. For a person of ordinary skill in the art, other changes or variations in different forms may also be made based on the foregoing description. All implementations cannot and do not need to be exhaustively listed herein. Obvious changes or variations that are derived there from still fall within the protection scope of the invention of the present invention.

1. A polypeptide, wherein the polypeptide is selected from:
(1), a polypeptide having a substitution mutation in at least one position selected from the group consisting of positions 16, 25, 29, 49 and 58, as compared with a natural C structural domain of a protein A as shown in SEQ ID NO.1; wherein,
the position 16 is subjected to a substitution mutation into leucine or valine;
the position 25 is subjected to a substitution mutation into lysine, arginine, histidine or tryptophan;
the position 29 is subjected to a substitution mutation into alanine, leucine or threonine;
the position 49 is subjected to a substitution mutation into arginine or histidine; and
the position 58 is subjected to a substitution mutation into glycine, isoleucine or alanine; or
(2) a polypeptide having at least 80% of sequence identity to the polypeptide in (1) and retaining substitution mutation in at least one position selected from the group consisting of positions 16, 25, 29, 49 and 58.

2. The polypeptide of claim 1, wherein the position 16 is substituted and mutated to leucine; and/or
the position 25 is subjected to a substitution mutation into lysine; and/or
the position 29 is subjected to a substitution mutation into alanine; and/or
the position 49 is subjected to a substitution mutation into arginine; and/or
the position 58 is subjected to a substitution mutation into glycine.

3. The polypeptide of claim 1, wherein the polypeptide has the substitution mutation in position 58, and has the substitution mutation in at least one position selected from the group consisting of positions 16, 25, 29 and 49; or
the polypeptide has the substitution mutation in position 49, and has the substitution mutation in at least one position selected from the group consisting of positions 16, 25, 29 and 58; or
the polypeptide has the substitution mutation in position 29, and has the substitution mutation in at least one position selected from the group consisting of positions 16, 25, 49 and 58; or
the polypeptide has the substitution mutation in position 25, and has the substitution mutation in at least one position selected from the group consisting of positions 16, 29, 49 and 58; or
the polypeptide has the substitution mutation in position 16, and has the substitution mutation in at least one position selected from the group consisting of positions 25, 29, 49 and 58; or
optionally, the polypeptide has substitution mutations at positions 16, 25, 29, 49 and 58; or
optionally, the polypeptide has substitution mutations at positions 16, 25, 49 and 58; or
optionally, the polypeptide has substitution mutations at positions 49 and 58; or
optionally, the polypeptide has substitution mutations at positions 29, 49 and 58; or
optionally, the polypeptide has substitution mutations at positions 16, 25 and 29; or
optionally, the polypeptide has substitution mutations at positions 16 and 25; or
optionally, the polypeptide has a substitution mutation at position 49; or
optionally, the polypeptide has a substitution mutation at position 25.

4. A fusion-type multimeric protein, wherein the fusion-type multimeric protein comprises the fusion-type multimeric protein formed by fusion of the polypeptide according to claim 1.

5. The fusion-type multimeric protein of claim 4, wherein the fusion-type multimeric protein further comprises polypeptide B and/or polypeptide Zmb; wherein
the polypeptide B satisfies any of the following conditions:
a. having an amino acid sequence as shown in SEQ ID NO.2; and
b. having an amino acid sequence of at least 80% sequence identity to the amino acid sequence as shown in SEQ ID NO.2 in a;
the polypeptide Zmb satisfies any of the following conditions:
c. inserting an amino acid sequence ML in at least one position of the amino acid sequence shown in SEQ ID NO.2 in a, wherein the amino acid sequence of the amino acid sequence ML is shown in SEQ ID NO.3; and
d. inserting an amino acid sequence ML in at least one position of amino acid sequence described in b;
optionally, the amino acid sequence of the polypeptide Zmb is shown in SEQ ID NO.4.

6. The fusion-type multimeric protein according to claim 4, wherein the polypeptide is designated polypeptide Cm; the fusion-type multimeric protein comprises at least one polypeptide Cm, and/or at least one polypeptide Zmb;
optionally, the fusion-type multimeric protein comprises 1 to 6 polypeptides Cm, and/or 1 to 4 polypeptides Zmb;
optionally, the fusion-type multimeric protein comprises 4 polypeptides Cm and/or 2 polypeptides Zmb.

7. The fusion-type multimeric protein according to claim 4, further comprising at least one functional polypeptide FLD, wherein the at least one functional polypeptide FLD satisfies any of the following conditions:
A. having an amino acid sequence shown in SEQ ID NO.5; and
B. having an amino acid sequence of at least 80% sequence identity to the amino acid sequence as shown in SEQ ID NO.5 in A;
optionally, the functional polypeptide FLD is located between the polypeptide Cm and the polypeptide Zmb;
optionally, the fusion-type multimeric protein comprises 4 polypeptides Cm, 1 functional polypeptide FLD and 2 polypeptides Zmb sequentially from N-terminal to C-terminal.

8. A biomaterial, wherein the biomaterial comprises any of the following:
1) a nucleic acid molecule encoding the polypeptide according to claim 1; optionally, the nucleic acid molecule is DNA or RNA;
2) an expression cassette, a recombinant vector, a recombinant microorganism or a transgenic cell line expressing the polypeptide according to claim 1;
3) an expression cassette, a recombinant vector, a recombinant microorganism or a transgenic cell line containing the nucleic acid molecule described in 1);
4) a recombinant vector, recombinant microorganism or transgenic cell line containing the expression cassette described in 2) or 3); and
5) a host cell containing the recombinant vector described in 2) or 3) or 4);
optionally, the recombinant vector is constructed from the Escherichia coli expression vector pET-30a(+);
optionally, the host cell is Escherichia coli, and optionally, the Escherichia coli is Escherichia coli BL21(DE3).

9. A method for detecting, separating or purifying antibody, wherein the fusion-type multimeric protein according to claim 4 is coupled with a chromatography medium vector to form an affinity chromatography medium for antibody detection, separation or purification.

10. An affinity chromatography medium, wherein the affinity chromatography medium comprises the fusion-type multimeric protein according to claim and affinity chromatography medium vector;
optionally, the affinity chromatography medium vector includes but is not limited to agarose gel, dextran, cellulose, high molecular polymer with hydroxyl groups or silica gel.

11. The polypeptide of claim 2, wherein the polypeptide has the substitution mutation in position 58, and has the substitution mutation in at least one position selected from the group consisting of positions 16, 25, 29 and 49; or
the polypeptide has the substitution mutation in position 49, and has the substitution mutation in at least one position selected from the group consisting of positions 16, 25, 29 and 58; or
the polypeptide has the substitution mutation in position 29, and has the substitution mutation in at least one position selected from the group consisting of positions 16, 25, 49 and 58; or
the polypeptide has the substitution mutation in position 25, and has the substitution mutation in at least one position selected from the group consisting of positions 16, 29, 49 and 58; or
the polypeptide has the substitution mutation in position 16, and has the substitution mutation in at least one position selected from the group consisting of positions 25, 29, 49 and 58; or
optionally, the polypeptide has substitution mutations at positions 16, 25, 29, 49 and 58; or
optionally, the polypeptide has substitution mutations at positions 16, 25, 49 and 58; or
optionally, the polypeptide has substitution mutations at positions 49 and 58; or
optionally, the polypeptide has substitution mutations at positions 29, 49 and 58; or
optionally, the polypeptide has substitution mutations at positions 16, 25 and 29; or
optionally, the polypeptide has substitution mutations at positions 16 and 25; or
optionally, the polypeptide has a substitution mutation at position 49; or
optionally, the polypeptide has a substitution mutation at position 25.

12. A fusion-type multimeric protein, wherein the fusion-type multimeric protein comprises the fusion-type multimeric protein formed by fusion of the polypeptide according to claim 2.

13. A fusion-type multimeric protein, wherein the fusion-type multimeric protein comprises the fusion-type multimeric protein formed by fusion of the polypeptide according to claim 3.

14. The fusion-type multimeric protein according to claim 5, wherein the polypeptide is designated polypeptide Cm; the fusion-type multimeric protein comprises at least one polypeptide Cm, and/or at least one polypeptide Zmb;
optionally, the fusion-type multimeric protein comprises 1 to 6 polypeptides Cm, and/or 1 to 4 polypeptides Zmb;
optionally, the fusion-type multimeric protein comprises 4 polypeptides Cm and/or 2 polypeptides Zmb.

15. The fusion-type multimeric protein according to claim 5, further comprising at least one functional polypeptide FLD, wherein the at least one functional polypeptide FLD satisfies any of the following conditions:
A. having an amino acid sequence shown in SEQ ID NO.5; and
B. having an amino acid sequence of at least 80% sequence identity to the amino acid sequence as shown in SEQ ID NO.5 in A;
optionally, the functional polypeptide FLD is located between the polypeptide Cm and the polypeptide Zmb;
optionally, the fusion-type multimeric protein comprises 4 polypeptides Cm, 1 functional polypeptide FLD and 2 polypeptides Zmb sequentially from N-terminal to C-terminal.

16. The fusion-type multimeric protein according to claim 6, further comprising at least one functional polypeptide FLD, wherein the at least one functional polypeptide FLD satisfies any of the following conditions:
A. having an amino acid sequence shown in SEQ ID NO.5; and
B. having an amino acid sequence of at least 80% sequence identity to the amino acid sequence as shown in SEQ ID NO.5 in A;
optionally, the functional polypeptide FLD is located between the polypeptide Cm and the polypeptide Zmb;
optionally, the fusion-type multimeric protein comprises 4 polypeptides Cm, 1 functional polypeptide FLD and 2 polypeptides Zmb sequentially from N-terminal to C-terminal.

17. A biomaterial, wherein the biomaterial comprises any of the following:
1) a nucleic acid molecule encoding the fusion-type multimeric protein according to claim 4; optionally, the nucleic acid molecule is DNA or RNA;
2) an expression cassette, a recombinant vector, a recombinant microorganism or a transgenic cell line expressing the fusion-type multimeric protein according to claim 4;
3) an expression cassette, a recombinant vector, a recombinant microorganism or a transgenic cell line containing the nucleic acid molecule described in 1);
4) a recombinant vector, recombinant microorganism or transgenic cell line containing the expression cassette described in 2) or 3); and
5) a host cell containing the recombinant vector described in 2) or 3) or 4);
optionally, the recombinant vector is constructed from the Escherichia coli expression vector pET-30a(+);
optionally, the host cell is Escherichia coli, and optionally, the Escherichia coli is Escherichia coli BL21(DE3).

18. An affinity chromatography medium, wherein the affinity chromatography medium comprises the fusion-type multimeric protein according to claim 5 and affinity chromatography medium vector;
optionally, the affinity chromatography medium vector includes but is not limited to agarose gel, dextran, cellulose, high molecular polymer with hydroxyl groups or silica gel.

19. An affinity chromatography medium, wherein the affinity chromatography medium comprises the fusion-type multimeric protein according to claim 6 and affinity chromatography medium vector;
optionally, the affinity chromatography medium vector includes but is not limited to agarose gel, dextran, cellulose, high molecular polymer with hydroxyl groups or silica gel.

20. An affinity chromatography medium, wherein the affinity chromatography medium comprises the fusion-type multimeric protein according to claim 7 and affinity chromatography medium vector;
optionally, the affinity chromatography medium vector includes but is not limited to agarose gel, dextran, cellulose, high molecular polymer with hydroxyl groups or silica gel.


SEQUENCE LISTING (7 sequences)

SEQ ID NO: 1 - protein, 58 aa, synthetic construct
ADNKFNKEQQNAFYEILHLPNLTEEQRNGFIQSLKDDPSVSKEILAEAKKLNDAQAPK

SEQ ID NO: 2 - protein, 58 aa, synthetic construct [sequence omitted]

SEQ ID NO: 3 - protein, 15 aa, synthetic construct
GGHLIAGGSDSERKE

SEQ ID NO: 4 - protein, 73 aa, synthetic construct
VDNKFNKEQQNAFYEILHLPGGHLIAGGSDSERKENLNEEQRNAFIQSLKDDPSQSANLLAEAKKLNDAQAPK

SEQ ID NO: 5 - protein, 14 aa, synthetic construct
SGDGDGDGSITSKG

SEQ ID NO: 6 - protein, 392 aa, synthetic construct
ADNKFNKEQQNAFYELLHLPNLTEKQRNAFIQSLKDDPSVSKEILAEARKLNDAQAPGADNKFNKEQQNAFYELLHLPNLTEKQRNAFIQSLKDDPSVSKEILAEARKLNDAQAPGADNKFNKEQQNAFYELLHLPNLTEKQRNAFIQSLKDDPSVSKEILAEARKLNDAQAPGADNKFNKEQQNAFYELLHLPNLTEKQRNAFIQSLKDDPSVSKEILAEARKLNDAQAPGSGDGDGDGSITSKGVDNKFNKEQQNAFYEILHLPGGHLIAGGSDSERKENLNEEQRNAFIQSLKDDPSQSANLLAEAKKLNDAQAPKVDNKFNKEQQNAFYEILHLPGGHLIAGGSDSERKENLNEEQRNAFIQSLKDDPSQSANLLAEAKKLNDAQAPK

SEQ ID NO: 7 - DNA, 1176 bp, synthetic construct
  source 1..1176: mol_type = other DNA
gcggataataaatttaacaaggaacagcagaacgcgttttacgagctcttacacctgccgaatctcaccgaaaaacagcgcaacgctttcatccagtctctgaaagatgatccctcggttagtaaagaaattctggcagaagcacgcaaacttaatgacgcacaggcccctaaggccgataacaaatttaacaaggaacagcagaacgcgttttacgaactgctgcacctgccgaacctgactgaaaagcagcgcaatgcattcattcaaagccttaaagatgatccgagcgtttcgaaagagatacttgcggaagctcgcaaactcaacgacgcccaagcgccgaaggctgacaacaaattcaataaagagcagcaaaatgccttctatgagctgttacacctcccgaacctgaccgaaaaacagcgcaatgccttcatccagtcactgaaagacgatccgagcgtctccaaagaaattctagccgaagcgcgtaaactgaacgatgcgcaagcccctaaagccgataataaattcaacaaagagcagcagaatgccttctatgaactgttgcatctcccgaacttaacagaaaaacaacgtaatgcctttatccagtcattgaaggatgacccaagtgtatccaaagaaattcttgcagaagcccggaaactgaacgacgcccaggcaccaggcagcggtgatggtgatggagacggctcgattacgtctaaaggcgtggacaataaatttaacaaagagcagcagaacgcgttttatgaaattttacatctaccgggcggtcatttaatcgcgggcggatccgactcggagcgtaaagaaaatttgaatgaagagcagcgaaacgcatttattcaaagcctgaaggatgatccgagccagtcagcgaacctgctggcggaagcaaagaaattaaatgatgcccaggcgccaaaagtggataataaattcaacaaagagcaacaaaacgcgttttacgaaatcttgcacttgcccgggggccatttaattgcgggtgggagcgactctgaaaggaaggaaaatcttaatgaggaacaacgtaacgcttttattcagagtctgaaggatgatccgagccaatcagcgaatctgctggcagaagcgaaaaaactgaatgatgctcaggcgcctaag